(19) United States
(12) Patent Application Publication
(10) Pub. No.: US 2011/0179716 A1
(43) Pub. Date: Jul. 28, 2011

(54) INDUSTRIAL PLANT MANUFACTURING ITS OWN FUEL

(75) Inventors: Pierre Jeanvoine, Saint-Germain-En-Laye (FR); David Galley, Paris (FR)

(73) Assignee: SAINT-GOBAIN GLASS FRANCE, Courbevoie (FR)

(21) Appl. No.: 13/054,400

(22) PCT Filed: Jul. 16, 2009
(86) PCT No.: PCT/FR09/51422

§ 371 Date: Apr. 14, 2011

(30) Foreign Application Priority Data
Jul. 18, 2008 (FR) 0854880

(51) Int. Cl. C10J 3/46; B01J 19/00; C12M 1/00; C10J 3/00

(52) U.S. Cl. 48/197 R; 422/129; 435/289.1

(57) ABSTRACT

The invention relates to a plant comprising an industrial manufacturing unit having a burner that burns a combustible fluid, the unit generating combustion flue gases containing CO₂, characterized in that the plant comprises a combustible fluid production unit fed with organic matter, which is decomposed in the production unit to said fluid, the combustible fluid production unit comprising a thermochemical gasifier that decomposes the organic matter by the latter reacting with an oxidizing gas comprising steam or oxygen or CO₂ so as to form the combustible fluid in gaseous form.

The invention relates to an industrial plant using organic matter such as biomass as energy source.

The invention proposes a technology aimed at supplanting the use of fossil energy in industrial processes, and lowering the emissions of CO₂ into the atmosphere and the cost of energy. In point of fact, with the objective of reducing the concentration of greenhouses gases in the atmosphere, industrial manufacturers are encouraged by an appropriate fiscal policy to use not fossil energy (from petroleum or natural gas), as this always returns more carbon and CO₂ to the surface of the Earth, but instead renewable fuel, such as biomass that absorbs CO₂ for its growth.

The industrial plant according to the invention comprises, on the one hand, a manufacturing unit comprising a combustion system (including at least one burner) using a combustible fluid, in particular of the gaseous fuel type, said manufacturing unit generating combustion gases, and, on the other hand, a combustible fluid production unit (which may in particular include a gasifier), which produces combustible fluid generated by decomposition of organic matter. The combustible fluid is conveyed to the manufacturing unit, where it is burnt in a burner. The fluid production unit comprises a gasifier, creating the combustible fluid in the form of gas, the manufacturing unit and the gasifier advantageously being close together so that the combustible gas generated in the fuel production unit is not stored but sent directly to the manufacturing unit. This avoids transporting material and heat losses. The distance between the manufacturing unit and the fuel production unit is preferably less than 10 km and even less than 5 km. Thus, the invention relates in the first place to a plant comprising an industrial manufacturing unit comprising a burner which burns a combustible fluid, said unit generating combustion flue gases containing CO₂, and a production unit for producing said combustible fluid, said production unit being fed with organic matter, said organic matter being decomposed in said production unit to said fluid.

The heat of the flue gases may be used to heat an element of the combustible fluid production chain, such as an organic matter drier or a bioreactor generating the organic matter or a boiler. Advantageously, a heat flux coming from the industrial manufacturing unit is used to supply the energy needed to accomplish the reactions (which may be endothermic) of gasifying or liquefying the organic matter.

The manufacturing unit may in particular be a glass furnace (for all glass applications: flat glass, hollow glass, fibers, etc.), an electricity generator, a metallurgical plant, etc. This manufacturing unit uses at least one burner for burning a combustible fluid (gas or liquid), it being possible in particular for said burner to be of the submerged burner or roof burner type.

The gasifier operates in a thermochemical mode. Depending on the thermochemical mode, the organic matter is decomposed at high temperature by a thermo-chemical process in a thermal gasifier. The chemical reactions take place by the organic matter reacting with an oxidizing gas comprising steam or oxygen or CO₂, usually between 800° C. and 1700° C. The combustible gas thus produced, also called “synthesis gas” or “syngas”, contains large proportions of carbon monoxide and hydrogen. It also generally contains methane. The sum of the molar percentages of hydrogen and carbon monoxide is generally at least 10%, even generally at least 30% or indeed even at least 35%. This combustible gas generally has a net calorific value of at least 1 MJ/Nm³ and even generally at least 5 MJ/Nm³ and possibly reaching even at least 10 MJ/Nm³, but is generally less than 30 MJ/Nm³. The organic matter may be a combustible solid or liquid such as biomass and/or waste, such as spent tires, plastics, automobile grinding residues, sludge, combustible substitute materials (called CSMs) or even household waste. The organic matter may be of biological nature or come from the agri-food industry. It may be animal meal. It may be terrestrial or aqueous biomass, in particular of the following types: straw, miscanthus stalks, seaweed, wood biomass, energy crops, vines, brushwood with a short growth cycle, etc. It may also be coal, lignite, peat, etc. It may be waste wood or waste paper from the papermaking industry. It may be an organic polymer, for example polyethylene, polypropylene, polystyrene, tire residues or automobile component grindings. The biomass may advantageously be a seaweed. This is because seaweed requires only sunshine (apart from exceptions), water, CO₂ and oligo-elements for its subsistence. Its growth may be extremely rapid (several harvests per year) and it can be grown in a suitable bioreactor without competing with food crops. The rate of growth of seaweed in a bioreactor may be greater than 50 times the rate of growth in nature. The growth of seaweed may be accelerated by increasing the amount of CO₂ in its immediate environment, and it is this property that is exploited in a bioreactor. The biomass is generally gasified after being dried and reduced to the correct particle size. As the case may be, it may then be liquefied.

Let us recall that, depending on the biochemical gasification mode (not used in the context of the present invention), a biomass is decomposed in a bio-gasifier at a temperature of generally between 10 and 80° C., preferably between 40 and 70° C. and more generally between 40 and 65° C. through the influence of bacteria. Decomposition in a bio-gasifier generally takes place in the absence of air. According to this mode, the combustible gas formed (which may be called biogas) contains methane. It also generally contains carbon dioxide. A biochemical gasification plant requires much more space than a thermochemical gasification plant. Moreover, gas production therein is also much slower.

The combustible fluid formed feeds the burner of the industrial manufacturing unit. Owing to the combustion of the combustible fluid (via the burner) in the manufacturing unit, the latter discharges flue gases that represent a substantial source of heat and a source of CO₂. To give an example, the flue gas leaving glass furnaces is usually at between 300 and 600° C. The flue gas heat may especially be used for participating in the operation of the thermochemical gasifier. In particular, since the gasifier operates on the principle of a reaction between steam and organic matter (in the case of syngas), the flue gas heat may be used to heat and vaporize water in a boiler before this water is sent to the gasifier. Some of this flue gas heat may also be used to dry a biomass intended for a gasifier. Because of its rate of operation, its operation at high temperature and its large heat requirement (thermochemical reactions are endothermic), the thermochemical gasifier lends itself well to the use of the substantial amount of heat immediately available in the combustion flue gases coming from the industrial manufacturing unit.

Thus, the invention relates in the first place to a plant comprising an industrial manufacturing unit having a burner that burns a combustible fluid, the unit generating combustion flue gases containing CO₂, characterized in that the plant comprises a combustible fluid production unit fed with organic matter, which is decomposed in the production unit to said fluid, the combustible fluid production unit comprising a thermochemical gasifier that decomposes the organic matter by the latter reacting with an oxidizing gas comprising steam or oxygen or CO₂ so as to form the combustible fluid in gaseous form.

The flue gases coming from the manufacturing unit may be sent into a bioreactor containing the organic matter which is of the plant matter type such as seaweed, said plant matter assimilating the CO₂ from the flue gases in order for it to grow, said plant matter then being sent to the combustible fluid production unit in order to be decomposed to the combustible fluid.

The organic matter may be at least partly converted to oil by a pyrolysis operation before being sent to the gasifier. Certain solid organic matter, especially of the biomass type, may in fact be converted to a viscous liquid (or oil) by pyrolysis at around 500° C. under pressure (in the manner of petroleum, which forms naturally from organic matter). In particular, seaweed lends itself very well to this conversion, since it is even possible to convert around 40% of the mass of certain seaweed into oil. This conversion to liquid has the advantage of considerably reducing the volume of material to be introduced into the gasifier. In addition, this condensed matter in oil form becomes easily transportable to the extent that the cost of transporting it then becomes reasonable. This is not the case with the starting biomass, which is generally too bulky in view of the energy that it provides. Thus, according to the invention, the combustible fluid production unit may comprise a pyrolysis reactor for liquefying the organic matter before it is fed into the thermochemical gasifier.

Depending on the industrial unit, this combustible liquid coming from the thermal conversion of organic matter, especially of the biomass type, could also be sent directly to the burner (without being gasified). In this case, the combustible fluid is a combustible liquid and the unit for producing said combustible fluid comprises this pyrolysis reactor in order to convert this organic matter to a relatively oily liquid. In particular, it would be possible for this liquid to be fed directly into a burner, whether submerged or not, of a glass furnace.

The flue gases leaving the manufacturing unit are also a substantial source of carbon dioxide. This carbon dioxide may be used for directly feeding a biomass being grown in a bioreactor. Specifically, according to one embodiment of the invention, the CO₂ leaving the industrial unit serves to grow the biomass by biological conversion of the CO₂ to organic matter. Such an operation is carried out in a bioreactor. In the case of a seaweed, the bioreactor contains water in which the seaweed grows. The CO₂ coming from the industrial unit is bubbled into this growth water. Thus, the CO₂ dissolves in the water and comes into direct contact with the seaweed, which can thus assimilate it. The bioreactor is thus connected to the heat/CO₂ flux output by the industrial manufacturing unit. The heat/CO₂ flux can therefore be used in a combined manner, by injecting the flue gases, or a portion thereof, directly into the bioreactor or, as the case may be, after purification and/or heat exchange so as to lower the temperature of flue gases. The sulfur possibly contained in the flue gases in the form of sulfates may also have a favorable role in the metabolism of certain types of biomass. The amount of CO₂ that can be recovered from the flue gases is equal to the amount needed to grow the biomass. The bioreactor is preferably located in the immediate vicinity of the industrial manufacturing unit so as to avoid having to transport material to prevent heat losses.

At least a portion of the heat/CO₂ flux leaving the glass furnace may therefore be used to grow the biomass necessary for providing the manufacturing unit with energy (complete integration of the energy chain into the industrial manufacturing line) or only to facilitate the treatment (drying, gasification, etc.) of a starting biomass external to the production line.

The mineral portion of the biomass (phosphates, potash, etc.) obtained after the gasification and/or liquefaction operation, for example in the form of ash, may be recycled into the bioreactors as nutrients for growth of the biomass.

The invention also relates to an industrial manufacturing process operated by the plant according to the invention. In particular, the industrial manufacturing unit may manufacture glass. This glass is melted in a furnace having a burner that burns the combustible fluid.

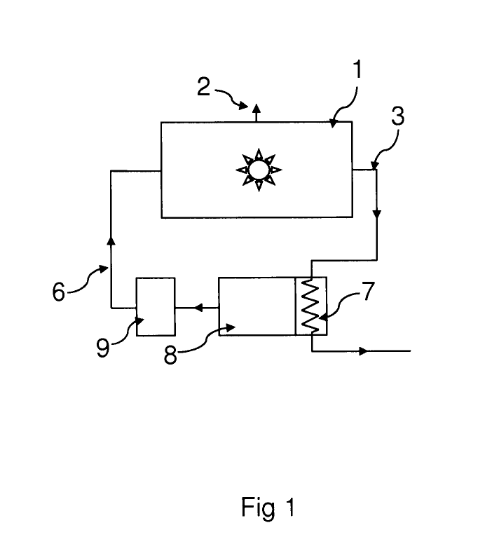

FIG. 1 shows a manufacturing unit 1, the output of which (for example glass) leaves at 2. Flue gases are generated by at least one burner in said unit and discharged at 3. These flue gases are sent to a heat exchanger 7 so that the heat of the flue gases is taken up by a bioreactor 8 in which seaweed is growing. This seaweed decomposes in a thermal gasifier 9, producing a combustible gas which is sent via 6 to the industrial manufacturing unit 1.

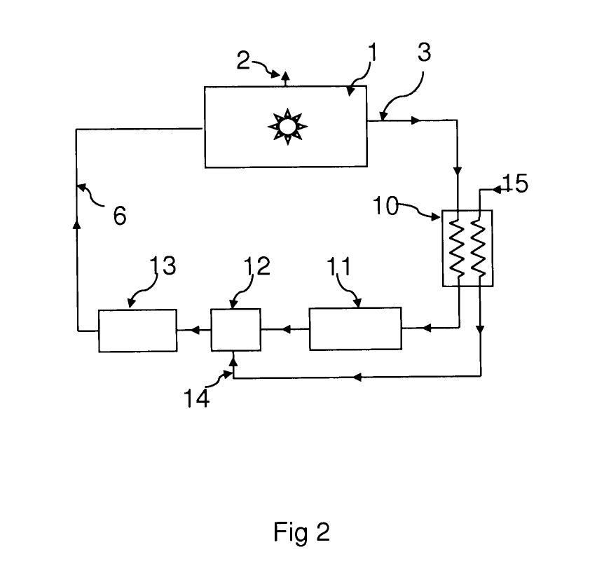

FIG. 2 shows a manufacturing unit 1 the output (for example glass) of which leaves at 2. Flue gases are generated by at least one burner in said unit and discharged at 3. The flue gases pass via a heat exchanger 10, so as to yield some of their heat, and then go directly into a bioreactor 11 in which seaweed is growing. The seaweed produced in the bioreactor 11 is then sent to a drier 12. In the heat exchanger 10, some of the flue gas heat has been taken up by an air circuit, which enters the heat exchanger at 15, and the hot air is sent via 14 to the drier 12 in order to dry the seaweed. The dried seaweed is then decomposed in a thermal gasifier 13 so as to produce a combustible gas which is sent via 6 to the industrial manufacturing unit 1.

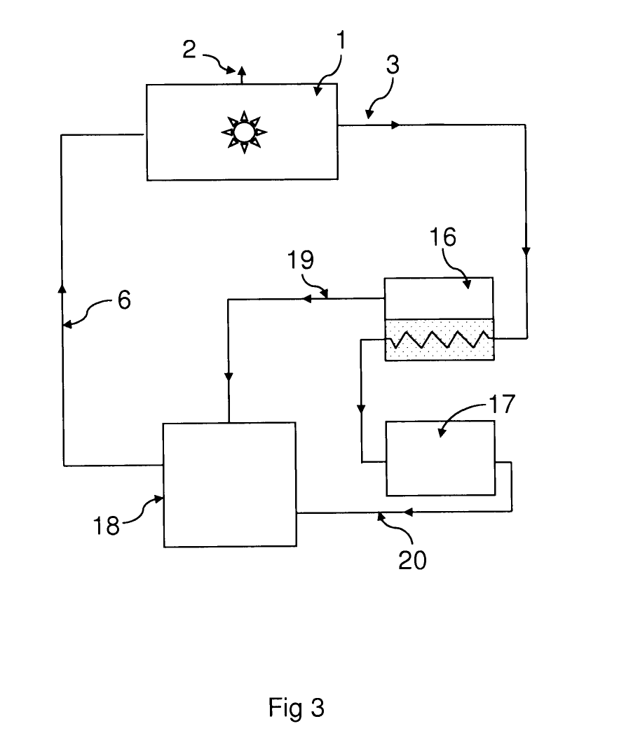

FIG. 3 shows a manufacturing unit 1 the output (for example glass) of which leaves at 2. Flue gases are generated by at least one burner in said unit and discharged at 3. The flue gases pass through a boiler 16 for heating water intended to be vaporized and are then sent to a bioreactor 17 in which seaweed is growing. The seaweed consumes the CO₂ from the flue gases in order to grow. This seaweed is then sent via 20 to a thermal gasifier 18 that produces a combustible gas which is sent via 6 to the burner of the industrial manufacturing unit 1. The steam created by the boiler 16 is sent via 19 to the gasifier so as to react with the biomass and produce the syngas.

### EXAMPLE

The case of a glass furnace of 30 megawatt power is considered. If the gasifier does not benefit from a return of energy coming from the furnace, the total amount of biomass needed for complete operation of the line is 80 000 t/year (at 4 MWh/t): this biomass supplies 240 000 m³/day of syngas with an NCV (net calorific value) of 3 kWh/m³ for feeding the glass furnace and 60 000 m³/day for operating the gasifier. The biomass represents about 150 000 t/year of CO₂ possibly available for feeding the growth of the biomass in the bioreactor. If the gasifier does benefit from a return of energy coming from the flue gases in the form of sensitive heat (4 MW available), it may be used (in the following nonlimiting list):
to dry the biomass in order to bring its moisture content to below 10%; and/or
to preheat the heat-transfer medium of a fluidized-bed or circulating-bed gasifier, thereby making it possible to save on the energy coming from the biomass and to increase the volume of gas available; and/or
to heat bioreactors in which the biomass is grown; and/or
to preheat the syngas for feeding the main furnace or the thermal gasifier.

1. A plant comprising an industrial manufacturing unit comprising a burner that burns a combustible fluid, the unit generating combustion flue gases comprising CO₂, wherein the plant comprises a combustible fluid production unit fed with organic matter, which is decomposed in the production unit to said fluid, the combustible fluid production unit comprising a thermochemical gasifier that decomposes the organic matter by reacting the organic matter with an oxidizing gas comprising steam or oxygen or CO₂ so as to form the combustible fluid in gaseous form.

2. The plant as claimed in claim 1, wherein the fluid production unit comprises an element heated by the heat of the flue gases.

3. The plant as claimed in claim 1, wherein the element is a drier for drying the organic matter.

4. The plant as claimed in claim 2, wherein the element is a bioreactor.

5. The plant as claimed in claim 2, wherein the element is a boiler.

6. The plant as claimed in claim 1, wherein the flue gases are sent into a bioreactor comprising the organic matter which comprises plant matter, said plant matter assimilating the CO2 from the flue gases in order for it to grow, said plant matter then being sent to the combustible fluid production unit in order to be decomposed to the combustible fluid.

7. The plant as claimed in claim 1, wherein the organic matter is seaweed or miscanthus.

8. The plant as claimed in claim 1, wherein the unit for producing said combustible fluid comprises a pyrolysis reactor for liquefying the organic matter before it is fed into the thermochemical gasifier.

9. An industrial manufacturing process operated by the plant according to claim 1.

10. The process as claimed in claim 1, wherein the industrial manufacturing unit manufactures glass.
